(19) United States
(12) Patent Application Publication
(10) Pub. No.: US 2016/0030723 A1
(43) Pub. Date: Feb. 4, 2016

(54) Multi-Chamber Anesthetic Delivery System

(71) Applicant: LANNETT COMPANY, INC., Philadelphia, PA (US)

(72) Inventor: Arthur P. Bedrosian, Pomona, NY (US)

(21) Appl. No.: 14/776,174

(22) PCT Filed: Mar. 17, 2014
(86) PCT No.: PCT/US14/30587

§ 371 Date: Sep. 14, 2015

Related U.S. Application Data
(60) Provisional application No. 61/789,923, filed on Mar. 15, 2013.

(51) Int. Cl. A61M 35/00

(52) U.S. Cl. CPC A61M 35/003; A61M 2202/048

(57) ABSTRACT

The present invention is directed to a multi-chamber delivery system of a topical anesthetic formulation for improving the stability of such anesthetic compositions. Topical anesthetics formulations have long been used for providing analgesia prior to any invasive medical procedure. Their use is essential for performing diagnostic, therapeutic, and cosmetic dermatology procedures. Topical anesthetics can be formulated in variety of dosage forms and mixtures such as solutions, creams, ointments, gels, and even patches and peels.

This application claims priority of U.S. provisional application Ser. No. 61/789,923 filed on Mar. 15, 2013.

### FIELD OF THE INVENTION

The present invention relates to a delivery of a topical anesthetic formulation to a subject in need thereof by employing a multi-chamber delivery system. The present invention also describes a method of improving the stability of such anesthetic formulations that are traditionally known to be susceptible to degradation.

### BACKGROUND OF THE INVENTION

Topical anesthetics formulations have long been used for providing analgesia prior to any invasive medical procedure. Their use is essential for performing diagnostic, therapeutic, and cosmetic dermatology procedures. Topical anesthetics can be formulated in variety of dosage forms and mixtures such as solutions, creams, ointments, gels, and even patches and peels. They can be applied to certain areas of the body including skin, nose, mouth to cause loss of feeling or numbness to allow medical practioners to perform their procedure. Another area of use of anesthetic formulations is in pain control. Pain relief is especially important among both pediatrics and geriatrics patients, where even minimal pain may result in an anxious and uncooperative patient.

Among topical anesthetics, cocaine has been the golden standard. Cocaine is a weakly alkaline compound and can be combined with acidic compounds to form various salts. The hydrochloride (HCl) salt of cocaine is by far the most commonly used in the art. Different salts dissolve to a greater or lesser extent in various solvents. The hydrochloride salt is polar and quite soluble in water. For medicinal use, cocaine is typically available in powder and solutions of various concentrations. The powder can be applied via moistened cotton swabs generally for nasal or oral numbness. The premade topical solutions are available in strengths ranging from 2-10%, with the 4% solution being the most common. Epinephrine may be added to a cocaine solution to limit systemic absorption while promoting vasoconstriction and enhanced local availability of the drug. To use cocaine in the nasal cavity, cotton applicators may be soaked in the cocaine solution and then applied to the area of interest and then removed via suitable forceps. The cocaine aqueous solution is recommended to be stored at room temperature between 68-77° F. (20-25° C.) away from light and moisture. Such solutions should not be frozen as it may impact the stability and efficacy of the product. Once the vial of an anesthetic solution is opened, many events can lead to product's waste. Such events include bacterial contamination; solvent evaporation, change in drug concentration and eventual drug degradation.

Aside from cardiovascular side effects, cocaine is associated with psychological dependence, which may lead to cocaine abuse and increased chance of serious side effects. Due to the cost, the side effect profile, the potential abuse and the limited shelf life stability, cocaine's use as a topical anesthetic has been falling out of favor among practioners. Accordingly, other combinations of anesthetic have been receiving some attention. Tetracaine, epinephrine, and cocaine (TAC) solution is a dermal anesthetic that is used for wounds, such as lacerations and abrasions, to provide anesthetic effect prior to wound repair. The original formulation of TAC solution consisted of tetracaine 0.5%, 0.05% epinephrine 1:2000, and 11.8% cocaine in normal solution. At this concentration, each mL of TAC solution contains 5 mg tetracaine, 0.5 mg epinephrine, and 118 mg cocaine.

Another common use of topical anesthetics is in dental applications. These products are frequently applied to the gum prior to the injection of any anesthetic. Transdermal anesthetics are also useful for numbing an area prior to venipuncture, such as blood drawing. Lidocaine and prilocaine are another type of anesthetic. They are both amide-type local anesthetic agents. Amides, are favorable as anesthetic agents, compared to esters, which are more sensitizing and can produce redness, swelling, irritation, itching, and other reactions.

Regardless of the type of the anesthetic compound, a frequent issue facing their manufacturers and medical practioners is how they can improve their shelf-life, because their availability is impacted by their relatively short shelf-life. Therefore, advance orders for manufacturing of anesthetic solutions coupled with subsequent storage of such products frequently leads to large amount of product waste. Accordingly, there is a need to facilitate a product that reduces abuse, while preserving stability of the product.

### SUMMARY OF THE INVENTION

The present invention addresses the need in the art. Broadly, at least one aspect of the present invention is directed to a multi-chamber delivery system for delivery of topical anesthetic formulations. In the preferred embodiments, the present invention is a two-chamber delivery system for improving the shelf life beyond the shelf-life of counterpart topical anesthetic formulations available in the art.

In at least one aspect of the invention, the topical anesthetic properties can be provided by a single anesthetic compound. In certain embodiments, the anesthetic properties are provided by a combination of anesthetic compounds present in the formulations. In at least one embodiment, the topical anesthetic formulation contains a secondary ingredient such as a vasoconstrictor, an anti-bacterial compound, a corticosteroid, or a non-steroidal anti inflammatory. In a preferred embodiment the anesthetic compound can be any one of tetracaine, lidocaine, benzocaine, dyclonine, pramoxine, dibucaine, butacaine, cocaine, mepivicaine, bupivicaine, levobupivicaine, ropivicaine, etidocaine, prolicaine, articaine, procaine, chloroprocaine, salts or other suitable pharmaceutically acceptable forms thereof including free acid or base, alone or in combination with each other.

In at least another aspect of the present invention, the first chamber of the described delivery system and the second chamber are connected through a narrow constricted neck. In yet another aspect of the invention, the neck is sealed by a septum plug. In one embodiment, one of the chambers contains an actuating stopper that seals the inside environment from the outside, effectively eliminating the access to the internal environment of the system. In yet another embodiment, the stopper may be of material that can act as an applicator. In yet another embodiment, the stopper can be actuated by downwards force being exerted thereon. In yet another embodiment, the septum plug can be displaced by actuating the stopper and applying pressure downwards on the stopper. In at least one embodiment, one of said chambers is of glass material. In another embodiment, the chambers may include a tamper resistant means for example, a protective member to be removed or at least transferred into a release configuration, thereby giving way to the actuating stopper.

In one embodiment, the removal or displacement of the protective member is only possible after a breakable seal is split open or destroyed. Thus, by coupling the protective member and the housing by means of a breakable seal, any tampering of the system can be detected.

In at least another aspect of the invention, the first chamber contains a plunger that is in directly contact with the septum plug located in the connecting neck. In one embodiment of the present invention pressing down on the plunger will displace the septum plug allowing free flow of the solvent through the connecting neck. In another embodiment, the top portion of the plunger is encircled by the stopper and the stopper can also act as an applicator. In yet another embodiment, the plunger is placed inside a cylindrical barrel that extends from the top of the first chamber to the septum plug.

In at least another aspect of the present invention, the first chamber and the second chamber can be separated. In such embodiment, the first chamber may be the upper chamber and carries the diluent, while the second chamber is the lower chamber containing the anesthetic ingredient. The chambers are sealed separately, yet can be connected to each other after removal of their respective seals, via a lock-and-seal mechanism. In this aspect of the invention, the lock and seal mechanism is designed to fit in the form of a narrowing neck between the chambers. In this aspect of the invention, the second chamber is connected through a turn and twist mechanism through a narrowing or a constricted neck forming the connection between the chambers. In yet another aspect of the invention, the neck is sealed by a septum that is placed at the bottom of the upper chamber. In another embodiment, an applicator can separately be placed on the lower chamber after the reconstitution of the formulation.

In at least one aspect of the invention, the lock-and-seal mechanism is the same as the turn and twist mechanism and is in the form of internal threaded system providing a mating engagement with threaded portions of the upper and lower chambers. Accordingly, the threaded system facilitates the coupling of the upper and the lower chambers. In a preferred embodiment, the upper chamber is rotated in a clockwise direction and the rotation bringing the chambers together and causes the sealing of the chambers from outside environment through their respective threads. In this aspect of the invention, the lower anesthetic containing chamber contains an applicator to be used for dabbing to the region of interest.

In accordance to another aspect of the invention, the lower chamber further contains a cap assembly to separately seal and cover the threaded region. The threaded region may further be adopted to carry an applicator located within a locking assembly. Such applicator will be sealed in a manner to stay sterile by a protective member or a tamper resistant means. The lower chamber can then be used as a reservoir to store the final solution and application of the anesthetic formulation. The applicator may further be capped to avoid contamination after use.

In another embodiment the anesthetic topical anesthetic formulation prepared by the two-chamber system is a cocaine solution. In such aspect of the invention, the two chamber system provides for a topical cocaine delivery system that contains a suitable solvent in one chamber and cocaine powder in another. In at least one embodiment of the present invention, the chamber is made of darker glass material to protect the anesthetic powder and/or the final solution from exposure to the sun light and potential degradation. At least one advantage of such glass material is that the cocaine powder can not be extracted from the chambers or is an abuse resistant formulation and therefore, the present delivery system offers less opportunity for improper appropriation of the cocaine powder.

The abuse resistant feature of this invention may be provided by incorporating surfactants, additives or stabilizers into the anesthetic mixture. In at least one embodiment, a suitable surfactant for example may be mixed with the anesthetic by the way of milling, blending, spray drying, coemulsifying, or melting of an additive, a surfactant, or a stabilizer with the anesthetic compound. In a more preferred embodiment, the additive, the surfactant, or the stabilizer may be adsorbed on the surface of the anesthetic particles, so that separating the anesthetic compound from such ingredients would not be readily achievable.

In another embodiment, the anesthetic compound is made more lipophilic by elimination reduction of the overall charge of the anesthetic compound. For example, a water soluble salt may be converted to a free base or free acid. In another embodiment, fatty acids or alcohols may be used to convert the water-soluble compound to a lipophilic construct. In yet another embodiment, the anesthetic compound may be coated with a water insoluble polymer which when exposed to the solvent can be removed by a separation mechanism such as a filter.

According to one aspect of the present invention, the delivery system is a two-chamber vial. In at least this aspect of the invention, an anesthetic liquid formulation is prepared in at least one of the chambers just prior to use. In another aspect of the invention, at least one of the chambers contains a plurality of anesthetic compounds in powder, crystal, particulate or nanocrystal forms and the other chambers contains a suitable solvent for dissolving such anesthetic compounds.

According to another aspect of the present invention, the delivery system is designed to stabilize the one or more anesthetic compound(s) prior to the topical application for at least 1, 2, 3, 4, 5, 6, 7, or 8 years, wherein less than 10% of the anesthetic compound is degraded during such period of time at temperature up to 40° C. In another embodiment the delivery system contains at least one anesthetic compound such as tetracaine, lidocaine, benzocaine, dyclonine, pramoxine, dibucaine, butacaine, cocaine, mepivicaine, bupivicaine, levobupivicaine, ropivicaine, etidocaine, prolicaine, articaine, procaine, chloroprocaine, salts or other suitable pharmaceutically acceptable forms thereof including free acid or base, alone or in combination with each other. That stays stable for at least 5, 4, 3, 2, or 1 year(s), wherein less than 10% of such compounds are degraded during such period of time at temperatures up to 40° C. According to another aspect of the present invention, the system is designed to keep the anesthetic formulation stable for at least 2, 3, 4, or 5 years at room temperature under atmospheric pressure. In another embodiment the shelf life stability of the delivery system is 3 years.

In another aspect of the present invention, the two-vial chamber delivery system contains no trace of bicarbonate. According to this aspect of the invention, the first chamber contains a pharmaceutically acceptable biocarbonate free incompressible liquid solvent; and the second chamber comprising a sterile solid particulate form of an anesthetic compound or a pharmaceutically acceptable salt thereof; a sealed septum separating the first and second chamber comprising material that is impermeable to said solvent, and an actuating stopper sealing the first or second chamber from the outside environment, wherein upon pressing said stopper, said incompressible liquid solvent is pressurized and drives said sealed septum downward, releasing the liquid solvent into the other chamber to form a mixture of liquid solvent and the anesthetic compound.

According to yet another aspect of the present invention, the system for releasing/delivering one or more active requires application of external force to the actuating stopper seal.

In yet another embodiment of the present invention, at least one of the anesthetic compounds in powder form is cocaine. In another embodiment, cocaine is the sole anesthetic compound present in the delivery system, the composition is free of any traces of bicarbonate. In another embodiment, the composition may contain bicarbonate.

In another aspect of the invention, the stopper material is of dehydrated water absorbable polymers or copolymers that would be converted to a ready to use applicator once hydrated with the solvent or mixture thereof. In one embodiment, the stopper material can also act as an applicator. In another embodiment, the applicator contains a solvent absorbable polymers and can be any one or combinations of sodium alginate, sodium carboxymethyl cellulose, sodium pectinate, sodium carboxymethyl chitosan, sodium polyacrylate, naturally occurring gums and synthetic polymers containing carboxylic acid, acrylic acid-methacrylic acid copolymers and/or dehydrated hydrogel, cross-linked macro-molecular network, fibers, nylons, rubber, cotton, and rayon, and mixtures thereof.

In another aspect of the invention, the stopper is an actuating stopper with a protruding means from at least one of the chambers, wherein said actuating means is a means for applying external pressure that is transferred via the liquid solvent in the first chamber to the sealed septum plug, and wherein said pressure disengages the plug from the constriction, thereby pushing the plug into the lower chamber to bring the solvent into contact with the solid particulates in the second chamber.

In another aspect of the invention, the product produced in the two-chamber delivery system is used for treating a skin, hair, ear, mucosal membrane, rectal, nasal or dental condition in a subject in need thereof the method comprising topically applying onto a skin, hair, ear, mucosal membrane, rectum, nose or tooth. Therefore another aspect of the present invention is directed to the use of the composition made by the two-chambered delivery system and topically delivering the composition. In yet another embodiment the delivery system contains an applicator.

According to still further features in the described preferred embodiments the final form of the composition is selected front the group consisting of an aqueous solution emulsion, an oil, a gel, a lotion, a suspension, a powder, an aerosol, a spray, and a foam. In on certain embodiment the present invention there is provided a method of delivering a topical anesthetic in the form of a solution, oil, lotion, suspension, aerosol, spray or foam made by the process for using the two-chamber delivery system. Another embodiment of the invention is directed to method of stabilizing an aqueous anesthetic composition using the presently described two-chambered delivery system.

In a preferred embodiment, the present invention is directed to a topical, transdermal anesthetic preparation comprising at least one of about 1-15% cocaine, 1-15% lidocaine; about 1-5%, prilocaine; about 0.1-1.0% dibucaine; with or without about 0.1-2.0% as effective for local vasoconstriction, of a sympathomimetic amine, preferably phenylephrine.

### BRIEF DESCRIPTION OF THE DRAWINGS

FIG. 1 is an enlarged side view of a two-chamber mixing delivery system that is an illustrative embodiment of the invention.

FIG. 2 is a side view of an alternative two-chamber delivery system.

FIG. 3 is a side view of another embodiment of the two-chamber delivery system.

### DETAILED DESCRIPTION OF THE INVENTION

Two-chamber mixing vials have been described in the art. At least one such vial is disclosed in U.S. Pat. No. 4,258,845 to Potts, incorporated herein by reference. The vials presented herein however are new for anesthetic formulations and are designed to increase shelf-life of such products beyond their commercially available counterparts.

At least one aspect of the present invention is directed to multi-chamber container containing separate chamber with thy anesthetic powder and at least another chamber containing a solvent. According to this aspect of the invention, additional chambers may be present to store a third ingredient or maintain additional space for mixing or withdrawal of the final mixture. According to this assembly, the desired material and measured quantities are placed in separate chambers.

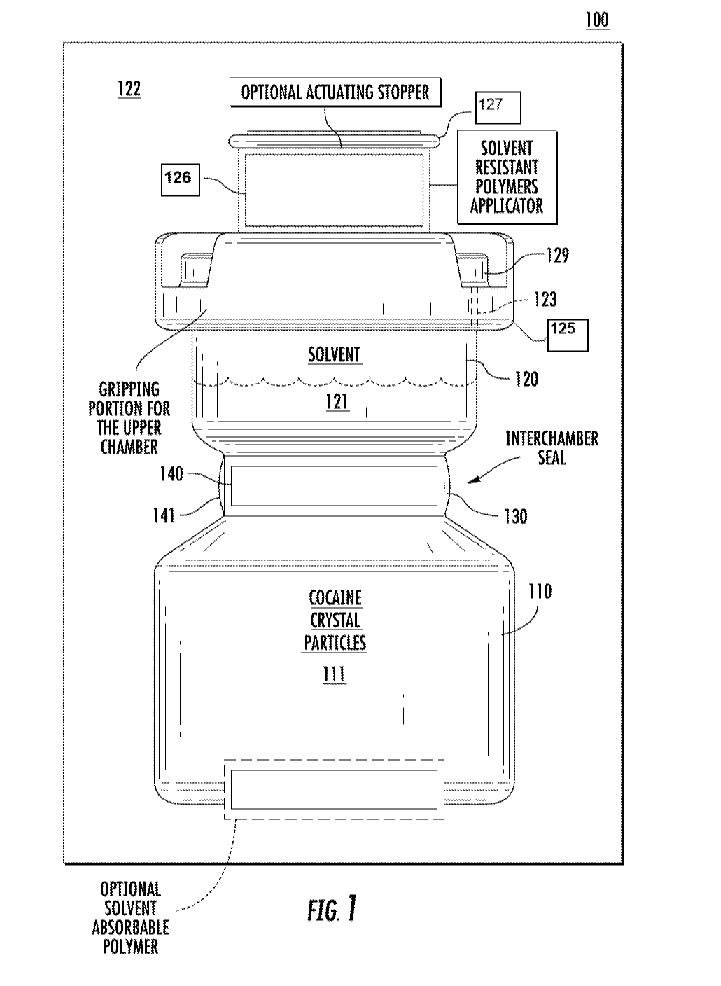

Another aspect of the present invention is directed to a two-chamber delivery system for delivery of topical anesthetic formulations. One such embodiment of the present invention is illustrative as two-chamber system 100, depicted here in FIG. 1. In a more preferred embodiment, the system is a vial of any suitable material such as glass, plastic, nylon, aluminum, but preferably glass and contains two interior chambers; a lower chamber 110 and an upper chamber 120 which are separated by a constriction 130. The constriction 130 is substantially airtight and watertight by a sealed septum plug 140. The septum can be made of any suitable material, but is preferably of rubber to maintain an airtight seal. The upper chamber 120 corresponds to the “first chamber” and the lower chamber 110 to the “second chamber.”

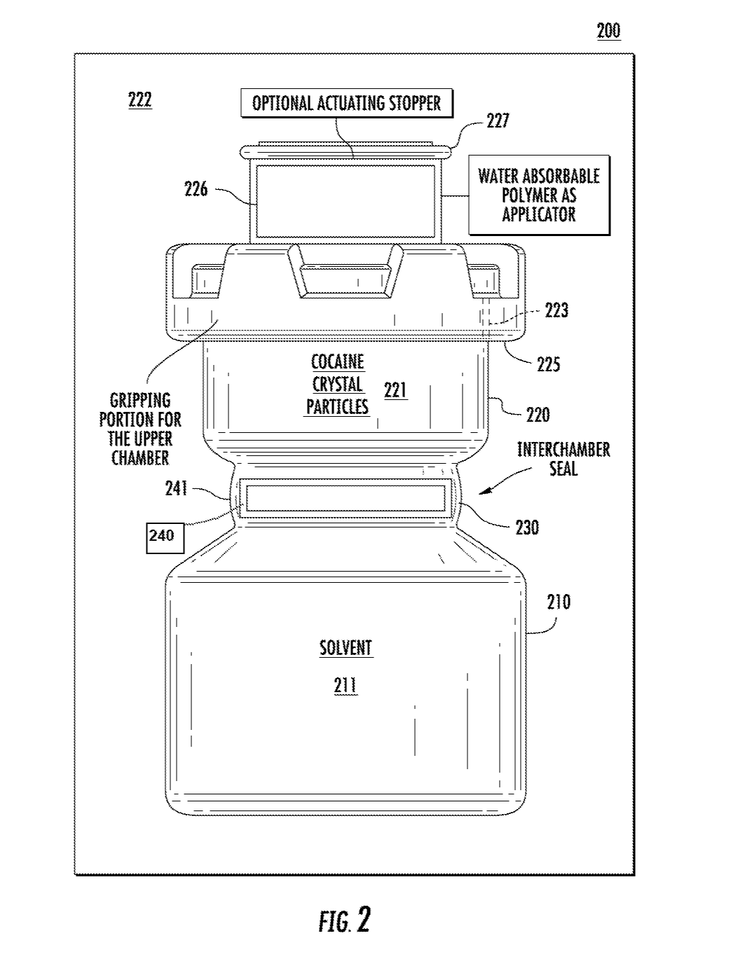

In at least one embodiment, the upper chamber 120 contains solvent 121. In another embodiment 200, the upper chamber 220 contains anesthetic compounds 221 in powder, crystal powder, microparticle, or nanoparticle forms and the rest of the headspace is filled with an inert gas that does not interact with the to avoid degradation of the anesthetic compounds, see FIG. 2. In one embodiment, the anesthetic properties are provided by a combination of anesthetic compounds present in the resulting formulations. In at least another embodiment, the topical anesthetic formulation contains a secondary ingredient such as a vasoconstrictor, an anti-bacterial compound, a corticosteroid, and a non-steroidal anti inflammatory, which can be in powder, microparticle, or nanoparticles residing in the first chamber.

In a preferred embodiment the anesthetic compound can be any one of tetracaine, lidocaine, benzocaine, dyclonine, pramoxine, dibucaine, butacaine, cocaine, mepivicaine, bupivicaine, levobupivicaine, ropivicaine, etidocaine, prolicaine, articaine, procaine, chloroprocaine, salts or other suitable pharmaceutically acceptable forms thereof including free acid or base, alone or in combination with each other. In more preferred embodiment the anesthetic is tetracaine, benzocaine, cocaine, bupivacaine, ropivicaine salts or other suitable pharmaceutically acceptable forms thereof. In another aspect of the present invention, anesthetic formulations are described that are produced by the instant process.

The septum plug 140 in FIGS. 1 and 240 in FIG. 2 corresponds to the sealing component placed within respectively the constrictions 130 and 230 separating the two chambers. The septum plug eliminates the availability of the content of the chambers to each other. In one embodiment, the septum plug prevents access to the anesthetic powder of the lower chamber until such septum is forcefully displaced or removed from the constriction 130. The septum plug forms an interchamber seal preventing moisture from traveling into the lower chamber inadvertently causing clumping of the anesthetic powder. In at least another embodiment, the upper chamber contains an actuating means 124 or 224. The actuating means is the component that would allow application of hydraulic pressure to the system and eventual displacement of the septum plug.

In at least one embodiment in FIG. 1, the actuating means contains the securing portion 125 and the stopper 126 and the protective member structure 127 located on top of the stopper. In at least another embodiment the actuating means is located on top of the upper chamber 120. In at least one embodiment, the user is able to press the actuating means and exert such pressure that is transmittable by the inert gas and the solvent within the upper chamber towards the neck of the vial and directly transferring the pressure to the septum plug 140 to dislodge the plug from the neck constriction 130 of the vial, pushing the plug into the lower chamber 110, thereby bringing the solvent of the upper chamber 121 into contact with the anesthetic powder 111 of the lower chamber.

In at least another embodiment the stopper is of solvent resistant rubber material. In yet another embodiment the stopper is of solvent permeable material which can be hydrated with the formulated mixture of solvent and the anesthetic compound(s) and then act as a topical applicator upon removal of the protective member 127. The applicator may then be separately capped or sealed to limit exposure to external contaminants.

In at least one embodiment, the actuating means contain the securing portion 125 on top of the upper chamber sealing the top chamber from the outside environment. In this embodiment, the securing portion is in the form of an assembly that covers the ledge, rim and the immediate region to facilitate proper sealing and further allow the stopper to move internally upon downward force. In a more preferred embodiment, the securing portion assembly is of a somewhat rigid material, typically a rigid plastic such as polyethylene, polypropylene, polyvinyl or acrylic material.

In yet another embodiment illustrated in FIG. 2, the actuating means contains the securing portion 225 and the stopper 226 and the closure protective member structure 227 located on top of the stopper. In at least this embodiment the actuating means is located on top of the upper chamber 220 which can be pressed down to actuate and exert pressure that is transmittable by the inert gas within the upper chamber towards the neck of the vial and directly transferring the pressure to the septum plug 240 to dislodge the plug from the neck constriction 230 of the vial, pushing the plug into the lower chamber 210, thereby bringing the solvent of the lower chamber 221 from into contact with the anesthetic powder 211 of the upper chamber.

In at least one embodiment, the upper chamber of the present delivery systems have a neck 123, 223, 323 which is the immediate region below the rim of the end of the upper chamber, and an access or the opening to the outside environment 122, 222, 322. The neck 123, 223, 323 in the illustrated embodiment is of substantially the same interior diameter as the upper chamber 120, 220, 320 but optionally the neck 123, 223, 323 can be of reduced diameter or be merged with the stopper assembly. The neck 123, 223, 323 may further have an outward projecting and or can be in touch with a plunger like assembly that can move freely within the neck region. In another embodiment, the neck may contain a lock-and-seal, or a twist-and-turn mechanism to facilitate separation of chambers from each other.

In at least another embodiment the upper chamber 120 is substantially filled with a liquid solvent for dissolving the anesthetic powder. In a more preferred embodiment, the solvent is bicarbonate free. In another embodiment, the upper chamber contains the anesthetic powder 221 and the liquid solvent is in the lower chamber 211.

In at least one embodiment, the neck 123 or 223 is provided with a securing portion in any structure or suitable design. In the illustrated embodiments the plug septum 140, 240, 340 is fabricated from flexible material such as elastomer that is impervious to aqueous solvents, such as water and/or gaseous substances such as air, nitrogen, or other inert gases such as helium, argon, xenon, radon, or radon or any combinations thereof. At least one example of such material is butyl rubber. The septum plug 140, 240, 340 has a sealing portion 141, 241, 341 seated within the constriction 130 or 230 between the two chambers. To improve the seal formed between the sealing portions 141, 241 of the septum plug and the constriction neck 140 or 240, there may be one to a plurality of spaced annular ridges, bulges or protrusions that can be fit within the structural design of the constriction.

In at least one embodiment, when the external force is applied downwards and internally to the actuating means and via the stopper 126 the generated pressure is transferred to the liquid solvent in the upper chamber 120 and subsequently transferred to the septum plug to be dislodged. In another embodiment, the external force is applied to the actuating means which subsequently increases the internal pressure of the upper chamber, thereby dislodging the septum plug. In another embodiment, the actuating means is connected to piercing column that can pierce through the septum plunge and create a hole, which upon retraction of the actuating means can allow free flow of the solvent through each of the chambers. In yet another embodiment, the actuating means is in the form of the plunger 330 that is in direct contact with the septum plug 350 and capable of dislodging the septum plug when direct forced is applied. In this embodiment, the plunger is placed inside a cylindrical barrel that extends from the top of the first chamber to the septum plug.

In a preferred embodiment, the upper opening of the upper chamber is sealed by protective means. Such structure contains the cap assembly 127, 227, the actuating stopper 126, 226 and the securing portion 125, 225. The securing portion 125, 225 surrounds the rim 129, 229 and the upper chamber neck or immediate proximate region of the same. The upper the ledge or the rim may contain an internal or external component that covers the upper chamber opening which has a diameter smaller than the upper open end, such that the pressing the actuating stopper inward would result in retaining of the stopper at the rim or in the upper chamber's neck.

The cap assembly 127, 227 may have an internal lock structure that acts with the stopper to prevent the stopper from being displaced downward relative to the middle of the upper chamber 120, 220. In at least one embodiment, the stopper is made of such material that can be rigid enough to withstand the external pressure and transfer the pressure inside. In another embodiment the stopper is capable of being hydrated with the solvent or the resulting anesthetic liquid mixture so that the stopper itself can act as a topical applicator.

A typical lock structure may contain a lock ring projecting inward from the lower end of the upper chamber to hold the stopper at the upper end of the upper chamber, a securing portion that extends outwardly to rim and the immediate upper region of the upper chamber, and a cap on top of the stopper to withstand external inward pressure, while prevent the stopper from moving to the middle of the upper chamber.

In at least another aspect of the present invention, the first chamber and the second chamber can be separated. In such embodiment, the first chamber 120 may be the upper chamber that carries the solvent or the diluent, while the second chamber 110 is the lower chamber containing the anesthetic ingredient. The chambers are sealed separately, yet can be connected to each other after removal of their respective seals, via a lock-and-seal mechanism. In this aspect of the invention, the lock and seal mechanism is designed to fit in the narrowing neck between the chambers. In this aspect of the invention, the first and second chambers are connected through a screw on mechanism through the narrow or the constricted neck between the chambers wherein the entire system will be locked and sealed from the outside environment. In yet another aspect of the invention, the neck is sealed by a septum that s placed at the bottom of the upper chamber. In an alternate embodiment, the applicator can separately be placed on the lower chamber after the reconstitution of the formulation. In another embodiment, the applicator may be sealed or covered by a cap to limit exposure to external contaminants.

In at least one aspect of the invention, the lock-and-seal mechanism is in the form of a twist and turn assembly. In yet another embodiment, the lock-and-seal mechanism is an internal threaded system providing a mating engagement with threads portions of the upper and lower chambers. Accordingly, the threaded system facilitates the coupling of the upper and the lower chambers. In one embodiment, the upper chamber is rotated in a clockwise direction and the rotation bringing the chambers together and causes the mating of the chambers through respective threads thereby facilitating the locking between the upper and the lower chambers. Accordingly, upon mixing the anesthetic compound and the solvent, the chambers are unscrewed and separated and the lower chamber containing the mixture is sealed with an applicator component that can be threaded into the lower chamber's threading system. Subsequently, the mixture may be applied to the region of interest via the applicator by dabbing the applicator to the region of interest. The applicator may further be sealed or capped to limit its exposure to external contaminants.

In accordance to another aspect of the invention, the lower chamber further contains a cap assembly to separately seal and cover the threaded system, wherein a lock latches and enable the lower chamber with an applicator located within the locking assembly would stay sterile. The lower chamber can then be used for direct dissolution and application of the anesthetic formulation. In this aspect of the invention, the lower chamber contains the anesthetic compound and the upper chamber contains the solvent and the actuating assembly. Accordingly, once the two-chamber system is assembled the actuating means of the upper chamber may be pressed down to dislodge the stopper positioned within the narrowing of the lower chamber. In one embodiment, the headspace in each chamber is filled with gaseous substances such as air, nitrogen, or other inert gases such as helium, argon, xenon, radon, or radon or any combinations thereof to keep the internal pressure constant for proper transfer of pressure.

In an alternative embodiment, the bottom threaded region of the upper chamber may be sealed by a cover and the upper threaded region of the lower chamber may be sealed by a removable cover. Accordingly prior to use, the sealed of each respective chambers are removed and the chambers are connected through their respective threaded assembly. The movement of the ingredients from one chamber to another can then be facilitated by pressing down the actuating means or via a piercing mechanism that allows connection of the upper to lower chamber. The piercing mechanism can be a protruding element or an inside hollow port within the one of the chambers threaded region that upon mating with the treaded region of the other chamber pierces through a seal or stopper, facilitating movement of solvent from upper chamber to the lower chamber.

In at least another embodiment, the two-system chamber assembly contains a first glass or plastic reservoir having a protective member covering at both ends, wherein at least one end contains an actuating means and the other end contains a cap sealed threaded region. In such embodiment, the at least second chamber has a threaded region that can mate with the threaded region of the first chamber facilitating a connection between the first and second chambers and transfer of material between the chambers.

In at least another embodiment the threaded sides of the chambers are protected by a tear-off type portion which can be removed from the respective chamber by pulling on a tear tab prior to the connecting of the first and the second chambers. In this embodiment, at least one of the upper or lower chambers contains external threads which are provided on glass or plastic reservoir and are exposed. The other chamber contains threaded ridges that can mate with the external threaded region of the upper or lower chamber sealing and locking the system yet allowing free transport of diluent from one chamber to the other.

The mixing of the solvent with the active anesthetic particles is facilitated by the locking of the two sets of internal threads from the respective chambers. The threads of each respective chamber are adapted to mate with external threads of the other chamber enabling interconnection of the chambers and the making the two-chamber delivery system. In at least one embodiment, the threaded system is located in the narrow neck of the delivery system.

In yet another embodiment, once the anesthetic mixture is prepared the chambers can be separated from each other. In such embodiment, a sterile applicator component prepared from suitable material and encased into a threaded assembly can be screwed on the top of the chamber carrying the anesthetic mixture. The anesthetic solution can then be administered h dabbing or wiping against the region of interest.

In at least one embodiment, to actuate the vial, the protective cap 127, 227 sitting on top of the stopper is pressed downward, for example with a thumb, thereby breaking the sealed but fracturable connection that can exist between the stopper and the upper chamber opening, moving the lower end face of the stopper 126, 226 inwards towards the center of the upper chamber until the stopper locking structure is engages, preventing the stopper to go any lower.

In at least one embodiment, once the stopper is pressed down words, an internal hydraulic pressure is created within in the upper chamber 120 which is respectively transferred to the septum plug. When the stopper is fully depressed, the created hydraulic pressure is respectively transferred to the septum plug dislodging the plug from its original position, into the middle of the bottom chamber allowing the solvent to reach the anesthetic powder crystals. Furthermore, the exchanged headspace from both of the chambers would provide sufficient space for shaking of the resulting mixture for creating a homogenous mixture.

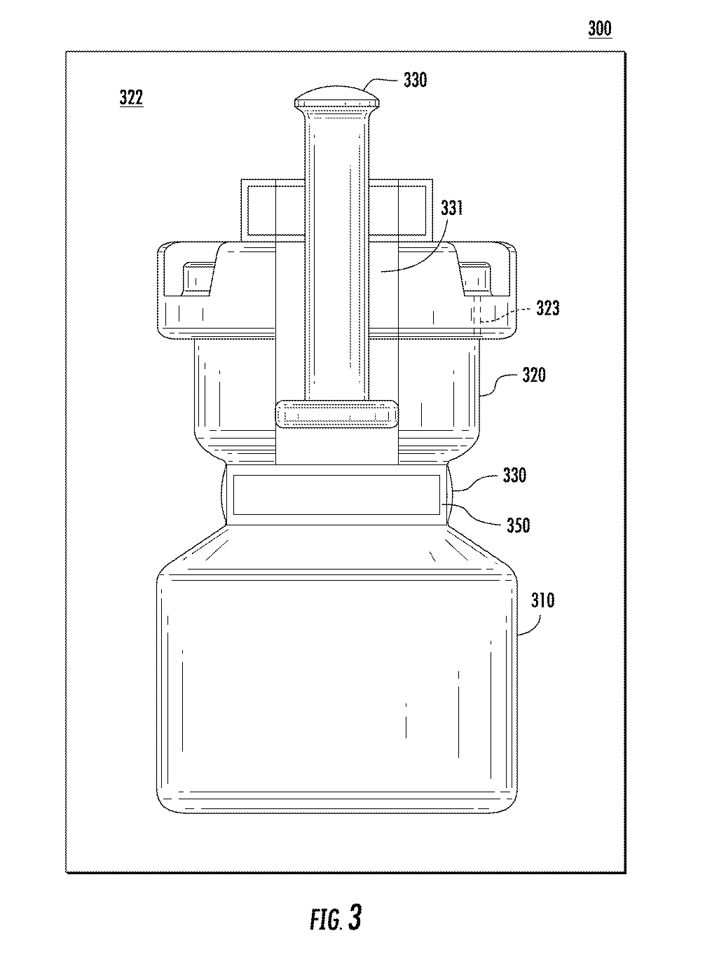

In yet another embodiment, the lower chamber contains the anesthetic powder and the actuating means is a plunger 330, FIG. 3. In such embodiment the lower chamber may have a third polymeric seal at the external end of the lower chamber that is capable of being saturated with the resulting mixture and act as a topical applicator for the proper region of the body. This polymeric seal can be a detachable component that can be separately assembled at the top of the lower chamber.

In yet another embodiment, the actuating plunger 330 will be pressed against the seal 350 to facilitate the movement of the solvent from the upper chamber to the lower chamber.

In another embodiment, to actuate the delivery system, the plunger 330 is pressed downward to directly dislodge the plug septum downward and facilitate flow of the solvent into the chambers. In this embodiment, the headspace in each chamber is filled with sufficient amount of gaseous substances such as air, nitrogen, or other inert gases such as helium, argon, xenon, radon, or radon or any combinations thereof to allow proper transfer of pressure from one chamber to another. In yet another embodiment, the stopper is connected to piercing columns that pierces through the septum plug when the stopper is pressed downwards. In such embodiment, the stopper may have a pull back mechanism to allow free flow of solvent into all chambers.

According to the present embodiment, the mixing of the solvent and the anesthetic compound may differ from that known in the art at least by having an anesthetic compound in either of the upper or lower chambers and an aqueous solution in a separate chamber. Should the either of the chambers as presently contemplated functions as a reservoir for air or other gaseous medium such nitrogen, or other inert gases such as helium, argon, xenon, radon, or radon or any combinations thereof, it provides a headspace for effective agitation following actuation of the vial. As such, by virtue of its lack of contact with the upper chamber prior to actuation, the system minimizes or prevents exposure of ingredients of the formulation to an environment that would have otherwise promoted instability and degradation.

In another embodiment, the small and inert headspace protects the formulation in the vial from oxidative degradation. The small treatment is indicative of conditions in an article of the present invention, wherein the formulation substantially fills the chamber in which it is packaged, with very little headspace, optionally containing inert gases such as helium, argon, xenon, radon, or radon or any combinations thereof, and therefore the system as a whole is capable of transferring any applied external force through to the septum plug.

In another aspect of the present invention a method of preparing anesthetic solution comprising providing a two-chamber vial delivery system comprising (a) an upper chamber comprising pharmaceutically acceptable incompressible liquid solvent, (b) a lower chamber comprising a sterile dry anesthetic compound or a pharmaceutically acceptable salt thereof (c) a seal separating the upper and lower chamber, and (d) a stopper sealing the upper chamber from the outside environment, pressing the stopper sealing the upper chamber, releasing the liquid solvent into the lower chamber to form a mixture, shaking the mixture to provide an anesthetic solution. In at least one embodiment, the solvent is bicarbonate free. In yet another aspect of the invention, anesthetic formulations are described wherein such formulations are made by a process described herein above.

Accordingly, in a particular embodiment, an article of manufacture of the present invention comprises any one of the anesthetic compound, an inert gas and a suitable solvent to dissolve the anesthetic compound substantially by agitating and mixing the vial content up and down. In a preferred embodiment the anesthetic is selected from any one of the following compounds tetracaine, lidocaine, benzocaine, dyclonine, pramoxine, dibucaine, butacaine, cocaine, mepivicaine, bupivicaine, levobupivicaine, ropivicaine, etidocaine, prolicaine, articaine, procaine, chloroprocaine, salts or other suitable pharmaceutically acceptable forms thereof including free acid or base, alone or in combination with each other. In a more preferred embodiment the anesthetic is tetracaine, benzocaine, cocaine, bupivacaine, ropivicaine salts or other suitable pharmaceutically acceptable forms thereof.

In another embodiment, solvent is any one of water, saline, 5% dextrose solution, any C₁-C₅ alcohol or any mixtures thereof. In yet another embodiment, the solvent is bacteriostatic and/or sterile. In another embodiment, the stopper system of the closure assembly in the upper chamber can act as a topical applicator once it is appropriately hydrated with the resulting mixture. In another embodiment, the applicator may be located in the lower chamber and capped with a secondary cap structure. In another embodiment, the secondary cap structure can contain a spaying means to deliver premeasured closes of the anesthetic.

In another embodiment, the formulation of the final product contains one or more additives such as a wetting and/or suspending agents in an amount effective to provide controlled flocculation of the drug, at least one of the wetting and/or suspending agents being susceptible to oxidative degradation; and, in a lower chamber thereof, only a gaseous medium, for example air or nitrogen, helium, argon, xenon, radon, or radon or any combinations thereof.

In a preferred embodiment of the present invention, at least the anesthetic compound of the present invention is cocaine as one of the anesthetic compounds. In another embodiment, cocaine is the sole anesthetic compound present in the delivery system.

In another aspect of the invention, the product produced in the two-vial chamber is used for treating skin, hair, ear, eyes, mucosal membrane, rectal, nasal or dental tissues in a subject in need thereof the method comprising topically applying onto a skin, hair, ear, mucosal membrane, rectum, nose or tooth. Therefore another aspect of the present invention is directed to the methods of use of the composition made by for example, a two-chambered delivery system directly to the site of interest in patients in need thereof.

In one embodiment, the stopper material is of dehydrated water absorbable polymers or copolymers that would be converted to a ready to use applicator once hydrated with the solvent or mixture thereof. In another embodiment, the stopper material can also act as an applicator. In another embodiment, the applicator contains a solvent absorbable polymers can be any one or combinations of sodium alginate, sodium carboxymethyl cellulose, sodium pectinate, sodium carboxymethyl chitosan, sodium polyacrylate, naturally occurring gums and synthetic polymers containing carboxylic acid, acrylic acid-methacrylic acid copolymers and/or dehydrated hydrogel, cross-linked macro-molecular network, fibers, nylons, rubber, cotton, and rayon and mixtures thereof. In another embodiment, the stopper material absorbs the mixture, containing the anesthetics in sufficient amounts to provide effective numbness in the area of interest.

In another embodiment of the present invention, the shelf life stability of the delivery system is at least 1 year, 2 years, 3 years, 4 years, 5 years but preferably up to at least 3 years and more preferably up to 5 years. In such assessment, the shelf-life stability is the degree of degradation of the original topical anesthetic compounds. In another embodiment, the stability is defined as any value that is higher than 15% percent degradation of the anesthetic compound during the storage period at temperature ranging from 15-28° C., but preferably at room temperature and atmospheric pressure. In a preferred embodiment, the corresponding anesthetic content does not degrade by more than 5% by weight of the initial amount after storage at room temperature for at least 5, 4, 3, 2, or 1 year(s), in the most preferred embodiment, the degree of degradation of the anesthetic is less than 1%

In one embodiment, the present invention, the system is designed to keep the anesthetic formulation stable for at least 2, 3, 4, or 5 years at room temperature under atmospheric pressure. In a most preferred embodiment, the content of the anesthetic compound does not degrade by more than 1% for at least 3 years.

According to an additional aspect of the present invention there is provided a method of delivering a topical anesthetic made by the process for using the two-chamber delivery system as well as methods of stabilizing an aqueous anesthetic composition using the presently described two-chambered delivery system.

According to still further features in the described preferred embodiments the final form of the composition is selected from the group consisting of an aqueous solution, emulsion, an oil, a gel, a lotion, a suspension, a powder, an aerosol, a spray, and a foam.

In a preferred embodiment, the present invention is directed to a topical, transdermal anesthetic preparation comprising (with all percentages being by weight) at least one of: about 1-15% cocaine, 1-15% lidocaine; about 1-5%, prilocaine; about 0.1-1.0% dibucaine; with or without about 0.1-2.0% as effective for local vasoconstriction, of a sympathomimetic amine, such as phenylephrine.

It will be apparent to those of skill in the art that many modifications can be made to the delivery system described immediately above without taking the final product outside the scope of the present invention. For example, the actuating means can comprise, in place of a means for applying hydraulic pressure to the contents of the upper chamber, a substantially rigid member that, when a downward force is applied to the cap assembly or a portion thereof, transmits the force directly to the septum or plug separating the upper and lower chambers. Applicator can be positioned in a manner to maximize integrity of the system without causing leakage or exposure of the product to the outside environment. Other two-chamber devices that can be substituted include those described, for example, in the patents individually listed below, each incorporated herein by reference. Other than the anesthetic compound can be in a piggyback structure of flexible polymeric structure.

To enhance the shelf-life stability, the drug particles are preferably very small, for example having a mean particle size smaller than about 0.01 microns to about 500 microns. It is sometimes desirable that the drug be micronized, i.e., reduced to an average particle size of about 1 to about 50 microns. Optionally all or a portion of the drug can be in nanoparticulate form, i.e., having an average particle size smaller than 1 microns.

In another embodiment, the product can contain at least one additive ingredient to act as wetting agent, suspending agents, emulsifier, surfactants, pH stabilizer, buffer, or other excipients in an amount effective to provide a product having a shelf life of at least 1 year, preferably at least 2, 3, 4, 5, 6, 7 or 8 years.

Example of such agents include without limitations polyethylene glycols (PEGs) with average molecular weight from about 100 to about 20,000, more typically about 200 to about 10,000. Suitable PEGs include PEG 2000, having an average molecular weight of 1800 to 2200, PEG 3000, having an average molecular weight of 2700 to 3300, PEG 3350, having an average molecular weight of 3000 to 3700, PEG 4000, having an average molecular weight of 3000 to 4800, and PEG 4600, having an average molecular weight of 4400 to 4800. Other agents further include poloxamers (polyoxyethylene-polyoxypropylene copolymers), illustratively of grades listed in the United States Pharmacopeia such as poloxamers 124, 188, 237, 338 and 407.

Emulsifiers or surfactants can include surfactants having a hydrophobic alkyl or acyl group, typically of about 8 to about 18 carbon atoms, and a hydrophilic polyoxyethylene chain. Preferred such surfactants are nonionic surfactants, illustratively including polyoxyethylene alkyl ethers such as laureth-9, laureth-23, ceteth-10, ceteth-20, oleth-10, oleth-20, steareth-10, steareth-20 and steareth-100; polyoxyethylene castor oil, polyoxyethylene hydrogenated castor oil, polysorbates such as polysorbate 20, polysorbate 40, polysorbate 60, polysorbate 65, polysorbate 80, polysorbate 85 and polysorbate 120; and polyoxyethylene alkyl esters, for example polyoxyethylene stearates. Polysorbates, for example polysorbate 80, are particularly preferred in such concentration as low as about 0.5 to about 10 mg/ml, typically about 1 to about 5 mg/mL.

The formulation can further contain antioxidants such as tocopherols (vitamin E), ascorbic acid (vitamin C) and salts and esters thereof, butylated hydroxytoluene (BHT), thiol derivatives including acetylcysteine, cysteine cystine, dithioerythritol, dithiothreitol, glutathione, methionine and thioglycerol, especially L-methionine, fimaric acid and salts thereof, hypophosphorous acid, malic acid, and L-methionine, typically in a total concentration of about 0.1 to about 50 mg/mL, preferably about 0.2 to about 20 mg/mL, and more preferably about 0.5 to about 10 mg/mL. Illustratively, L-methionine can usefully be present at a concentration of about 1 to about 5 mg/mL.

The formulation optionally further comprises a chelating agent. Optionally, the formulation can comprise, in addition to components described hereinabove, excipients such as those mentioned below. One or more additional wetting and/or suspending agents, can optionally be present. Such agents include polyvinylpyrrolidone (PVP), for example PVP having a molecular weight of about 2,000 to about 54,000, such as PVP K12, K17, K25 and K30, and surfactants such as phospholipids (e.g., lecithin), cationic surfactants (e.g., myristyl .gamma.-picolinium chloride), anionic surfactants (e.g., sodium lauryl sulfate), etc. One or more thickening or viscosity adjusting agents can optionally be present, for example cellulosic polymers (e.g., methylcellulose, carboxymethylcellulose, hydroxyethylcellulose, hydroxypropylmethylcellulose), gelatin and gums (e.g., acacia).

One or more preservatives can optionally be present, for example phenol, chlorobutanol, benzyl alcohol, methyl paraben, propyl paraben, sodium benzoate, benzalkonium chloride and cetylpyridinium chloride. One or more tonicity adjusting agents can be present, for example sodium chloride, sodium sulfate, dextrose, mannitol and glycerol. One or more buffering agents can optionally be present, for example buffers derived from acetic, aconitic, citric, glutaric, lactic, malic, succinic, phosphoric and carbonic acids. Typically such a buffer is an alkali or alkaline earth metal salt of such an acid. Phosphate and citrate buffers such as sodium phosphate and sodium citrate are preferred. In another embodiment, the buffering system may comprise sodium or potassium bicarbonate, chloride, acetate salts to alleviate pain at the site of injection.

Preferably the final composition of the invention has a pH of about 3 to about 7.5. An advantage of the invention is that pH of the composition can often be controlled within a narrower range. For example, in a cocaine composition as described herein, pH can typically be controlled within a range of about 3 pH to about 7.5 more preferably within a range of about 4 to about 7. In one embodiment, the composition comprise a blend of cocaine, a buffer system, and a bicarbonate salt.

In a preferred embodiment the final formulation contains an anesthetic compound in concentrations ranging from about 0.1% to about 25% weight. In another embodiment, such a concentration ranges from about 0.5% to about 15%. In a more preferred embodiment, such concentration is from 0.75% to 10%. For example, at least one such formulation is a 4% cocaine solution prepared immediately prior to use and had at least a 3 year shelf life before being reconstituted into the final solution. Solubility in water for many pharmaceutically useful compounds can be readily determined from standard pharmaceutical reference available in the art. The most preferred embodiment of the present invention is to providing stable formulations for compounds that are very soluble in the intended solvent system employed in the art. Cocaine HCl is the prime candidate of such compounds. Typically the cocaine salt powder used in the delivery system is highly solubility in water, for example having a solubility of at least 10 mg/mL or more.

In one embodiment, the anesthetic is cocaine to be administered topically. In this case, the cocaine is cocaine hydrochloride USP in crystalline, granular or powder form having a saline, slightly bitter taste that numbs tongue and lip. In a more preferred embodiment cocaine is present in concentrations of about 0.1% to about 25% in the final mixture. In a more preferred embodiment the concentration of the anesthetic is up to about 15% in the final mixture. In a more preferred embodiment, the solution may contain citric acid and sodium benzoate. In a more preferred embodiment, the external surface of unopened delivery system may be sterilized by ethylene oxide, but not steam autoclave.

In another embodiment, cocaine is present in a concentration of about 0.001 to about 50 mg/mL, preferably about 0.01 to about 10 mg/mL. In the case of tetracaine, the concentration of the active ingredient is between 0.1 to about 10 mg/mL, preferably about 0.5 to about 10 mg/mL. When both cocaine and tetracaine are present, the concentrations of the individual drugs are typically as given above. In the case that epinephrine is added to the powder, it can be in concentration of about 0.001 to about 2 mg/mL, preferably about 0.1 to about 0.2 mg/mL.

In another embodiment, the present system is formed in a chamber having a volume ranging from 0.5 mL to 50 mL. In one embodiment, the system offers a volume of about 10 mL dispensable as a solution delivered by in the form of a spray.

In yet another embodiment, the present system generates air anesthetic formulation free of any preservative consist essentially of a sterile anesthetic compound such as the cocaine, a stabilizer, and a solvent. In yet another embodiment, the system provides for a formulation that only consists of the anesthetic compound such as cocaine, an additive, a stabilizer, and the suitable solvent.

The present delivery system offers less opportunity for improper appropriation of the cocaine powder. In at least one embodiment of the present invention, the chamber is made of darker glass material to protect the anesthetic powder and/or the final solution from exposure to the sun light and potential degradation.

The abuse resistant feature of this invention may be provided by incorporating surfactants, additives or stabilizers into the anesthetic mixture. In at least one embodiment, a suitable surfactant for example may be mixed with the anesthetic by the way of milling, blending, spray drying, coemulsifying, or melting a surfactant, an additive or a stabilizer with the anesthetic compound. In a more preferred embodiment, the surfactant, additive or stabilizers can be adsorbing on the surface of the anesthetic particles, so that separating the anesthetic compound from such ingredients would not be readily achievable.

In another embodiment, the anesthetic compound is made more lipophilic by elimination reduction of the overall charge of the anesthetic compound. For example, a water soluble salt may be converted to a free base or free acid. In another embodiment, fatty acids or alcohols may be used to convert the water-soluble compound to a lipophilic construct. In yet another embodiment, the anesthetic compound may be coated with a water insoluble polymer.

According to one aspect of the present invention, the delivery system is a two-chamber vial. In at least this aspect of the invention, an anesthetic liquid formulation is prepared in at least one of the chambers just prior to use. In another aspect of the invention, at least one of the chambers contains a plurality of anesthetic compounds in powder, crystal, particulate or nanocrystal forms and the other chambers contains a suitable solvent for dissolving such anesthetic compounds.

In another embodiment, the chambers may include a tamper resistant means for example, a protective member to be removed or at least transferred into a release configuration, thereby giving way to the actuation means. In one embodiment, the removal or displacement of the protective member is only possible after the breakable seal is split open or destroyed. Thus, by coupling protective member and housing by means of a breakable seal, a tamper-evident closure means can be provided for a two-chamber system indicating, whether the system has been tampered or not.

In yet another aspect of the present invention, the anesthetic composition of the present invention contains an anesthetic selected from any one of the following compounds tetracaine, lidocaine, benzocaine, pramoxine, dibucaine, butacaine, cocaine, mepivicaine, bupivicaine, levobupivicaine, ropivicaine, etidocaine, prolicaine, articaine, procaine, chloroprocaine, salts or other suitable pharmaceutically acceptable forms thereof including free acid or base, alone or in combination with each other. In a more preferred embodiment the anesthetic is tetracaine, benzocaine, cocaine, bupivacaine, ropivicaine salts or other suitable pharmaceutically acceptable forms thereof and at least any one of the following secondary agents such as epinephrine, a stabilizer such as bicarbonates, a pH adjuster including suitable acids or bases as described above and oxymetazoline, or xymetazoline or both oxymetzoline and xymetazoline.

### EXAMPLE

The following examples illustrate aspects of the present invention but are not to be construed as limitations.

### Example 1

Samples of commercial cocaine topical formulation were prepared according to the process below. The cocaine delivery system of the present invention contained an upper chamber containing saline, and a lower chamber containing a dry mixture of cocaine HCl salt, sodium benzoate and citric acid formulation respectively in amounts, cocaine 4%, sodium benzoate 0.01 and citric acid 5%.

In this embodiment, the lower chamber and upper chamber of delivery system are separated at the center with a septum plug that is inserted into the neck constriction that exist between the two chamber and the solvent is placed in the upper chamber 120, 220, 320, all in a well-known manner. During the manufacturing process, the stopper is inserted into the neck of the upper chamber so that the enlarged stopper portion seals and engages the inner wall of the opening in the upper chamber. The cap and the securing system is positioned on the assembly so as to secure the stopper and the chambers themselves

At least one embodiment of the present invention is shown in FIG. 1. In this embodiment, the protective means is positioned so that the stopper portion 126 is received within the upper chamber. During the initial mounting of the protective means on the delivery system, the lower edge of the securing portion 125 is deformed so as to pass over the rim 129 and the cap snaps inwardly beneath the rim to be secured to the upper chamber. The stopper is thus positioned as illustrated in Figure, wherein the upper end of the stopper portion 126 is spaced downwardly from the upper end of the actuating means.

When it becomes desirable to use the cocaine product, the two-chamber vial is gripped within the hand so that the thumb can press against the upper end of the upper chamber. By urging the stopper toward the vial with the thumb, the connection between the tip of the stopper and the upper chamber is initially fractured and the stopper moves downwardly toward the center of the upper chamber. During this initial movement of the stopper, a locking mechanism at the upper neck may be activated along the stopper's ending until a locking ring engages in the manner to halt the stopper from going down any further. Continued downward depression of the sleeve portion may result in the stopper being pressed downwardly into the vial would not likely create any more hydraulic pressure than what has already been created within the upper chamber respectively forcing the septum plug 140, 240 out of the neck constriction 130 or 230 so that the solvent can move into the lower chamber and mix with the anesthetic dry powder.

When the stopper 126, 226 is depressed, it dislodges interchamber seal allowing the cocaine powder to mix with the aqueous solution. One advantage associated with this method of forming a cocaine solution is that the aqueous phase can be instilled first and sterilized separately for example via autoclaving or other means. This will prevent potential microbial growth in the aqueous phase prior to sterilization. In the case of using a plunger instead of a stopper, the depression of the sleeve plunger 331 passes through the opening in the upper chamber and causing the dislodging of the interchamber septum. Once the plunger is hilly depressed as indicated above, the solvent and the anesthetic powder can be freely mixed to provide a ready to use solution.

Various embodiments of the present invention provide surprising advantages. A spray dried starch formulations provide prolonged in-vial stability, particularly when the molecular weight of the starch is over about 500,000. The use of two-chamber vials with water providing an additional seal for the formulation and provide increased shelf life, and greater use convenience.

In another embodiment the contents of one commercial product vial were transferred into an applicator to provide a large surface area of application. In another embodiment, the contents of the commercial product vials were prepared into a single kit or box for delivery that can sit on the shelf for at least 3 years without losing any more than 10% of the active ingredient and/or any bacterial contamination.

Although a particular preferred embodiment of the invention has been disclosed in detail for illustrative purposes, it will be recognized that variations or modifications of the disclosed apparatus, including the rearrangement of parts, lie within the scope of the present invention.

What is claimed is:

1. An anesthetic delivery system comprising
a. A first chamber comprising pharmaceutically acceptable biocarbonate free incompressible liquid solvent,
b. A second chamber comprising a sterile solid particulate form of an anesthetic compound or a pharmaceutically acceptable salt thereof.
c. A sealed septum separating the first and second chamber comprising material that is impermeable to said solvent, and
d. An actuating stopper sealing the first or second chamber from the outside environment.
wherein upon pressing said stopper, said incompressible liquid solvent is pressurized and drives said sealed septum downward, releasing the liquid solvent into the other chamber to form a mixture of liquid solvent and the anesthetic compound.

2. The system of claim 1, wherein the anesthetic compound is selected from the group consisting of a group consisting of tetracaine, lidocaine, benzocaine, dyclonine, pramoxine, dibucaine, butacaine, cocaine, salts or other suitable pharmaceutically acceptable forms thereof.

3. The system of claim 1, wherein the stopper (d) is of a dehydrated water absorbable polymer or copolymer comprising sodium alginate, sodium carboxymethyl cellulose, sodium pectinate, sodium carboxymethyl chitosan, sodium polyacrylate, naturally occurring gums and synthetic polymers containing carboxylic acid, acrylic acid-methacrylic acid copolymrts and/or dehydrated hydrogel, cross-linked macro-molecular network, fibers, nylons, and mixtures thereof.

4. The system of claim 3, wherein the stopper is saturated with said mixture.

5. The system of claim 1, wherein the shelf life stability of the delivery system is at least 1 year.

6. The system of claim 1, further comprising an applicator component within at least one of said chambers.

7. The system of claim 1, said chambers are separated by a constriction wherein the septum is held in place in a form of a substantially airtight and solvent-tight plug.

8. The system of claim 7, wherein the chambers are connected via an annular neck and the septum is seating within the neck's sealing portion and an upper protruding portion that projects beyond the of the neck.

9. The system of claim 7, wherein the actuating stopper has a protruding actuating means from at least one of the chambers, wherein said actuating means is a means for applying external pressure that is transferred via the liquid solvent in the first chamber to the sealed septum plug, and wherein said pressure disengages the plug from the constriction, thereby pushing the plug into the lower chamber to bring the solvent into contact with the solid particulates in the second chamber.

10. A method of preparing anesthetic solution comprising
providing a two-chamber vial delivery system comprising (a) an upper chamber comprising pharmaceutically acceptable biocarbonate free incompressible liquid solvent, (b) a lower chamber comprising a sterile dry anesthetic compound or a pharmaceutically acceptable salt thereof, (c) a seal separating the upper and lower chamber, and (d) a stopper sealing the upper chamber from the outside environment,
pressing the stopper sealing the upper chamber,
releasing the liquid solvent into the lower chamber to form a mixture,
shaking the mixture to provide a anesthetic solution.

11. A method of treating a patient in need with an anesthetic formulation comprising
administering an anesthetic solution to said patient wherein said anesthetic solution is prepared by (a) providing a two-chamber vial delivery system comprising an upper chamber comprising pharmaceutically acceptable biocarbonate free incompressible liquid solvent; a lower chamber comprising a sterile dry anesthetic compound or a pharmaceutically acceptable salt thereof; a seal separating the upper and lower chamber; and a stopper sealing the upper chamber from the outside environment, (b) pressing the stopper sealing of the upper chamber, (c) releasing the liquid solvent into the lower chamber to form a mixture of anesthetic and solvent, (d) shaking the mixture to form the anesthetic solution, and
topically applying said anesthetic solution to an area in need thereof.

12. The method of claim 7, wherein upon the shaking of the mixture, the system stopper absorbs the anesthetic solution.

13. The system of claim 1, wherein the first chamber is the upper chamber and the second chamber is the lower chamber.

14. The system of claim 1, wherein the climbers are free of any preservatives.

15. The system of claim 1, wherein the anesthetic is present in an amount of about 1 to about 5 mg/mL.

16. The system of claim 1, wherein the formulation comprises: (a) cocaine or a pharmaceutically acceptable salt thereof in amounts 10-1000 mg/mL; (b) solvent; (c) a stabilizer and (d) optionally pH adjuster.

17. The system of claim 16, wherein the formulation consist essentially of (a) cocaine, (b) a stabilizer, and (c) solvent.

18. The system of claim 16, wherein the formulation consist of: (a) cocaine, (b) a stabilizer, and (c) solvent.
